SEQUENCE LISTING (3 sequences)

SEQ ID NO: 1 - DNA, 33 bp, Artificial
  Serotype 2 Dengue DNA primer
ggttggatgcgcgcatctattctgacccactgg

SEQ ID NO: 2 - DNA, 27 bp, artificial
  primer for RNA
atacaatgtggcatgtcacacgtggcg

SEQ ID NO: 3 - DNA, 30 bp, artificial
  probe complimentary to Zika RNA
aaccttcgctctattctcatcagtttcatg

(19) United States
(12) Patent Application Publication
(10) Pub. No.: US 2017/0107565 A1
(43) Pub. Date: Apr. 20, 2017

(54) FUNCTIONALIZED PARTICLES FOR LABEL-FREE DNA IMPEDIMETRIC BIOSENSOR FOR DNA AND RNA SENSING

(71) Applicant: Purdue Research Foundation, West Lafayette, IN (US)

(72) Inventors: Esteban Ernesto Marinero-Caceres, West Lafayette, IN (US); Richard J. Kuhn, West Lafayette, IN (US); Lia Antoaneta Stanciu, West Lafayette, IN (US); Seon-Ah Jin, West Lafayette, IN (US)

(73) Assignee: Purdue Research Foundation, West Lafayette, IN (US)

(21) Appl. No.: 15/297,776

(22) Filed: Oct. 19, 2016

Related U.S. Application Data
(60) Provisional application No. 62/243,879, filed on Oct. 20, 2015.

(51) Int. Cl. C12Q 1/68; G01N 27/02; G01N 27/327; C12Q 1/70

(52) U.S. Cl. CPC C12Q 1/6837; C12Q 1/701; G01N 27/026; G01N 27/3276

(57) ABSTRACT

In one embodiment, 3-Aminopropyltriethoxysilane (APTES) functionalized graphene oxide (APTES-GO) wrapped SiO₂ particle composite (SiO₂@APTES-GO) was prepared via the self-assembly process of APTES-GO sheets and SiO₂ particles. Transmission electron microscopy (TEM) and Attenuated Total Reflectance-Fourier Transform Infrared spectroscopy (ATR-FTIR) confirmed wrapping of the SiO₂ particles by the APTES-GO sheets. A biosensor based on electrochemical impedance spectroscopy (EIS) was constructed and used to sensitively detect dengue DNA and dengue RNA via primer hybridization using different oligonucleotide sequences. The results demonstrated that the SiO₂@APTES-GO electrode material led to enhanced sensitivity, selectivity and detection limit, compared to both APTES-GO and APTES-SiO₂. The three-dimensional structure, high surface area, electrical properties and the ability for rapid hybridization offered by the SiO₂@APTES-GO rendered this electrode material as ideal to use in the reported dengue impedimetric sensor.

### CROSS REFERENCE

This application claims the benefit of U.S. Provisional Application 62/243,879, filed Oct. 20, 2015, the content of which is expressly incorporated herein entirely.

### TECHNICAL FIELD

The present disclosure generally relates to biosensors, and in particular to methods and electrochemical sensors for detecting existence of viral diseases transmitted by insect sources.

### BACKGROUND

This section introduces aspects that may help facilitate a better understanding of the disclosure. Accordingly, these statements are to be read in this light and are not to be understood as admissions about what is or is not prior art.

Insect-borne viral diseases are a problem on a global scale. Vector monitoring, control, and eradication is essential in combating the world-wide impact of neglected tropical diseases (NTDs). For example, dengue fever is one of the most important arthropod-borne viral diseases that can lead to complications such as dengue hemorrhagic fever (DHF), dengue shock syndrome (DSS), and has the capacity to rapidly spread once a viral outbreak is established (Guzman and Harris, 2015). Tropical and subtropical areas in South America, Africa and South-East Asia are especially affected by dengue virus (DENV), which is spread by mosquitoes. Moreover, global warming, intercontinental transportation, and international travel are transforming DENV from a once limited regional problem into a global one. According to WHO (World Health Organization) about 40% of world's populations is at risk of Dengue and it is presently endemic in over 100 countries. The CDC (Center of Disease Control) estimates that as many as 400 million people are infected yearly. In addition, bacterial diseases spread by insects such as Lyme disease, malaria, leishmaniasis and Chagas disease exert a global toll on human health and economic development.

Early diagnosis of dengue is crucial to providing supportive medical treatment, especially in the case of hemorrhage and shock from DHF and DSS, and is especially important as there is no effective vaccine to prevent the dengue virus infection. A recent large test trial on the CYD-TDV vaccine for dengue did not provide the anticipated results. The vaccine failed to meet targeted levels of efficacy, especially for the dengue serotype 2, which was the predominant serotype in the test.

Current DENV detection methods rely on complex polymerase chain reaction (PCR) and enzyme-linked immune-sorbent assay (ELISA). However, performing these tests require high time investment, and meticulous specimen preparation. Additionally, the PCR method is prone to fallacious results due to contamination. Although the ELISA method is less complicated than PCR, it requires several days between fever symptom emergence and diagnosis because it is based on the detection of immunoglobulin (Ig) in blood. Thus, the test cannot be conducted until either antibodies such as IgM or IgG are produced in response to infection.

Recently, Zika fever (also known as Zika virus disease) brought more attention in the medical field. Zika fever is caused by the Zika virus. Most Zika virus cases have no symptoms, but when present they are usually mild and can resemble dengue fever. Symptoms may include fever, red eyes, joint pain, headache, and a maculopapular rash. Symptoms generally last less than seven days. It has not caused any reported deaths during the initial infection. However, infection with Zika virus during pregnancy causes microcephaly and other brain malformations in some babies, and infection in adults has been linked to Guillain-Barré syndrome (GBS).

Diagnosis of Zika virus infection is done by testing the blood, urine, or saliva for the presence of Zika virus RNA when the person is infected.

Prevention of Zika virus involves decreasing mosquito bites in areas where the disease occurs, and proper use of condoms. Efforts to prevent bites include the use of insect repellent, covering much of the body with clothing, mosquito nets, and getting rid of standing water where mosquitoes reproduce. There is no effective vaccine for the Zika virus yet. Health officials recommended that women in areas affected by the 2015-16 Zika outbreak consider putting off pregnancy and that pregnant women not travel to these areas. More importantly, early detection of Zika virus presence in the environment will provide guided control.

There is therefore an unmet need for methods and devices that can detect, monitor, and control insect-borne diseases.

### SUMMARY

This disclosure in general relates to a biosensor platform that is configured to provide impedimetric data in the presence of an insect-borne virus. The biosensor comprises an electrode material coupled to at least one functionalized particle, a supporting membrane, wherein the electrode material is disposed on the supporting membrane; and a label-free moiety immobilized to the electrode material. The supporting membrane may be a rigid substrate. The functionalized material may be graphene. The functionalized material may be positively charged. The label free moiety may be DNA, RNA or any other analyte that can be recognized by the insect-borne virus component. A non-limiting theory is that the immobilized moiety binds to the targeted virus component and triggers an impedimetric change in the sensor, such impedimetric change may be captured and recorded via transmission device and global position system. A preferred embodiment is a virus DNA or RNA as the immobilized moiety, but others like antibody, glycoprotein are within the contemplation of the disclosure. The virus may be dengue, yellow fever, chikungunya, West Nile and Zika virus.

This disclosure provides a biosensor platform to detect at least one vector borne virus. The biosensor platform comprising: a functionalized electrode surface, at least one nucleotide primer immobilized on the functionalized electrode surface, wherein the nucleotide primer is diagnostic for the at least one vector borne virus DNA or RNA; and an electrochemical impedance spectroscope (EIS), wherein the EIS is configured to measure the impedance change upon the primer hybridization to the virus DNA or RNA.

In one preferred embodiment, the aforementioned functionalized electrode surface comprises an electrode material deposited on a supporting membrane.

In one preferred embodiment, the aforementioned electrode material is a conductive material.

In one preferred embodiment, the aforementioned supporting membrane is a functionalized graphene sheet.

In one preferred embodiment, the aforementioned functionalized electrode surface is 3-Aminopropyltriethoxysilane (APTES) functionalized graphene oxide (APTES-GO) wrapped SiO₂ particle composite (SiO₂@APTES-GO).

In one preferred embodiment, the aforementioned biosensor platform further comprises a microfluidics device to extract the at least one vector borne virus's nucleotides for hybridization.

In one preferred embodiment, the aforementioned biosensor platform further comprises a wireless data transmission device and a power source.

In one preferred embodiment, the aforementioned biosensor platform further comprises a global position system coupled to the wireless data transmission device.

In one preferred embodiment, the aforementioned power source is solar.

In one preferred embodiment, the aforementioned virus to be detected is selected from the group consisting of dengue, yellow fever, chikungunya, West Nile and Zika viruses.

This disclosure further provides a method for detecting at least one vector borne virus. The method comprising:
a. Providing a biosensor platform comprising at least one nucleotide primer immobilized on a functionalized electrode surface, and the nucleotide primer is diagnostic for at least one vector borne virus DNA or RNA;
b. Contacting the biosensor platform with a sample;
c. Observing the vector borne virus specific impedance change to identify the presence of said at least one vector borne virus.

In some embodiment, the aforementioned method of detecting vector borne virus is configured for point of care detection with additional wireless data transmission device, power source and global position system to transmit the virus infection data from predetermined location to a control center.

In some embodiment, the aforementioned method of detecting vector borne virus uses a sample from human blood, and the human having been bitten by an arthropod species. The arthropod species may comprise mosquitoes, ticks, triatomine bugs, sandflies and backflies.

In some embodiment, the aforementioned method of detecting vector borne virus may include multiple viruses at the same time by making the platform an electrochemical biosensor array, wherein the array comprises a plurality of functionalized particles loaded with specific primer/probes to detect a plurality of vector-borne diseases.

These and other features, aspects and advantages of the present invention will become better understood with reference to the following figures, associated descriptions and claims.

### BRIEF DESCRIPTION OF THE FIGURES

FIG. 1 ζ potential distribution of (a) SiO2, (b) APTES-SiO2, (c) GO, (d) APTES-GO, and (e) SiO2@APTES-GO

FIG. 2 TEM image of as-prepared (a) SiO2 particle, and (b) SiO2@APTES-GO composite

FIG. 3 Interfacial charge-transfer resistance from hybridized complementary DNA (RCT-COM) of (a) APTES-GO, (b) APTES-SiO2, and (c) SiO2@APTES-GO composite

FIG. 4 ΔRCT of SiO₂@APTES-GO composite versus complementary and non-complementary target concentration (1) 10 pM non-complementary (COM) DNA, (2) 10 pM complementary DNA, (3) 1 fM complementary DNA, (4) 1 aM complementary DNA

FIG. 5 ΔRCT versus complementary RNA (RCT-COM) target concentration (1) 10 pM complementary RNA of APTES-SiO2, (2) 1 aM complementary RNA of APTES-SiO2 (3) 10 pM complementary RNA of SiO2@APTES-GO composite, and (4) 1 aM complementary RNA of SiO2@APTES-GO composite

FIG. 6: Impedance measurement for RNA dengue sensor based on graphene coated particles. Blue: before 1 fM RNA hybridization; green: after 1 fM RNA hybridization.

FIG. 7: Impedance response upon exposure of graphene-wrapped silica electrodes functionalized with DNA primers to solutions of 1 pM Zika RNA. The impedance increase indicates detection of Zika RNA.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In response to the unmet need, a novel method and bio-sensing platform and biosensor are presented herein. We have developed a biosensor platform for the detection of the dengue virus and other mosquito borne viral diseases. The invention employs functionalized graphene oxide wrapped silica particles and impedance sensing to selectively detect Dengue virus or other insect borne virus cDNA and RNA in solutions down to laM concentration.

Presently, arthropod-borne virus detection (Dengue, Zika, Chikungunya and West Nile virus etc.) rely on enzyme-linked immune-adsorbent assays (ELISA) and sensitive polymerase chain reaction (PCR) testing. These tests require complex and time consuming sample preparation, sample transport to specialized laboratories with trained personnel, and are also susceptible to false positive results. There are currently no point of care devices for the detection of the Zika virus (or any other arthropod-borne viral disease) offering in-situ, rapid, ultra-sensitive, accurate sensing capabilities for both personal and clinical use. This disclosure is to provide a biosensing technology that can be readily implemented for various insect borne virus detection, including various dangerous viruses such as Zika virus, west Nile virus etc. The biosensor detection can be integrated into inexpensive platforms for personal and clinical point of care applications. Equipping the device with easy to interpret output monitoring and WI-FI data sending capabilities, both the end user and the health care team can instantaneously respond to the test results.

Biosensors are bioanalytical tools that measure the presence of analytes by combining the sensitivity of biomolecular recognition elements with a physical transduction mechanism. They play a major role in the development of time-effective, low-cost and easy-to-use analytical tools and are particularly suitable for miniaturization and portability. Their advantages include their high sensitivity and specificity provided by the biocatalytic or biorecognition sensing elements. Various kinds of biosensors (enzyme-based, immunosensors, DNA-sensors) have been broadly studied but only few of them have been successfully commercialized. The global biosensors market is expected to grow from $6.72 billion in 2009 to $22.5 billion in 2020. Most of the developed biosensors address medical needs and are used for diagnostics purposes. Applications in environmental and agricultural fields, and particularly for anti-terrorist activity and homeland security, are also rapidly increasing. For example, optical biosensors have now the highest sensitivity, approaching theoretical limits of interface sensitivity, which is critical for detection of drug candidates, viruses, or pathogens.

Electrochemical biosensors function on the basis of correlating the electronic signal given off upon interaction of the biological recognition element with the analyte. There are different types of electrochemical biosensors, which measure the electrical properties of an electrode surface and the binding kinetics of molecules. In particular, electrochemical impedance spectroscopy (EIS) can measure the changes of the electrical properties of a surface arising from the interaction with the captured analyte, while minimizing sample damage during measurements.

Disclosed herein is a method of detecting, monitoring, and ultimately controlling the occurrence of insect-borne viral diseases. These viral diseases can include but are not limited to dengue, yellow fever, chikungunya, West Nile diseases and Zika fever. Also disclosed herein is a biosensor platform and biosensor system for accomplishing this detection and monitoring of insect-borne viral diseases. The method for detecting and monitoring insect-borne diseases, includes placing a biosensor in a predetermined location. This predetermined location can be chosen, as an example, by a user based on the presence of insects that are vectors for disease transmission. A sample of nucleic acid (NA) is obtained from such insects. The sample of NA is placed on a sensing device, and the sensing device is configured to measure a change in resistance to an applied electrical current when the NA sample corresponds to the virus-causing disease. The sample of NA (DNA or RNA) is probed and utilized in the sensing device to obtain impedance and resistance data. The impedance and resistance data can be used to determine if a particular virus is present in the insect. These results can be transmitted to the user, and the user (for example a disease control center or medical monitoring facility) can then utilize the data to take preventative measures such as selective fumigation. Such measures can be rapidly implemented to prevent further occurrences of such diseases by destroying the infected viral disease vectors.

There are different methods for obtaining the NA sample. For example, the sample of NA can be obtained from a person's blood sample, the individual having already been bitten by the insect. The sample of NA from the insect can also be obtained by enticing the insect to bite a membrane. The membrane can be configured to extract the virus sample and therefore its RNA from the insect through conventional microfluidics devices.

Also disclosed herein is a biosensor platform. The biosensor platform includes an electrode material configured to be coupled to at least one functionalized particle. The platform can host functionalized particles for detecting different viral RNA. The biosensor platform can also feature the electrode material disposed on the screen printed supporting strip. In one embodiment, the supporting strip can be a rigid substrate.

The functionalized material consists of positive functional groups on the surface of the material. The material can be an electrically conducting material. This electrically a conducting material can be graphene. The electrode material can be conductive material such as platinum, copper etc. The functionalized material can be silicon dioxide. Alternative materials that can be used are conductive materials that have high surface areas, such as for example conducting polymers such as poly(3,4-ethylenedioxythiophene) (PEDOT) or poly(p-phenylene sulfide (PPS), or functionalized metallic nanoparticles (e.g. silver), or silver nanoparticle loaded graphene.

In another embodiment, the biosensor can be implemented into a biosensor system, which has at least one functionalized material and a nanoparticle-based amperometric biosensor. The nanoparticle-based amperometric biosensor can be configured for nucleic acid (NA) purification and extraction from a grinded insect sample and subsequent probing of the NA to be compared to the genome of the disease causing virus. A wireless data transmission device can be coupled to the biosensor system, and further coupled to a power source. In yet another embodiment, at least one probe is coupled to the biosensor. These probes can be configured to allow detection of a particular virus. The biosensor system can also have a global positioning system (GPS). The GPS can be coupled to the wireless data transmission device to permit transmission of location data. The power source can be a battery. The power source in yet another embodiment can be configured to be self-sustaining, for example, it can be configured to run on solar power, and thereby permit unmanned and remote operation.

### Example 1: A Functionalized Graphene Based Particle for DNA Detection

### Material Preparation of Example 1

SiO₂ Particle Preparation:

Silica particles were synthesized by the modified Stober method (Lei et al., 2014): 9.01 ml of DI water, 50 ml of ethanol (100%, KOPTEC) and 1.37 ml of ammonium hydroxide (NH₃ 28˜30%, Sigma-Aldrich) were mixed together and 3.2 ml of tetraethyl orthosilicate (TEOS, 99%, Fluka) was added drop-wise into the mixed solution. After 1 hour, the synthesized particles were separated from the mixed solution using an ultracentrifuge (Eppendorf AG 22331, Hamburg, Germany) spinning at 14.5 krpm, and then repeatedly washed using ethanol at least six times. The washed particles were first dried at 354K for 6 hr and then were grinded into fine particles. Subsequently, they were heat-treated in air at 383K for 24 hr. The end product was finally grinded again.

Graphene Oxide Synthesis:

The graphene oxide (GO) sheets were prepared through chemical oxidation of graphite particles by a modified Hummer's method (Hummers and Offeman, 1958). Graphite powder, 0.85 gr, (99.9995%, Alfa Aesar) and 23 ml of H₂SO₄ (95˜98%, Sigma-Aldrich) were stirred for 8 hrs. Next, 3.0 g of KMnO₄ (≧99%, Sigma-Aldrich) was slowly added at a temperature below 294K. The mixture was then heated at 314K while constantly stirring it for thirty minutes, and subsequently for an additional 45 minutes at 344 K. The solution was next diluted with 46 ml of deionized water (DI) and heated at 373K for 30 min. The oxidation reaction was terminated by adding 140 ml of DI water together with 10 ml of H₂O₂ solution (30%, Macron) after being cooled down to room temperature. The oxidized graphite particles were washed and filtered several times using a 10% HCl (37%, Sigma-Aldrich) solution with DI water, then dried at 333 K under vacuum. Exfoliation was conducted to ultimately synthesize GO sheets by using a bath sonicator (Cole-Parmer 8891; Cole-Parmer, Vernon Hills, Ill., USA) and a Branson digital sonifier 102C (Branson, Danbury, Conn., USA).

Positively Charged Graphene Oxide Preparation:

Positively charged graphene oxide was prepared using 3-Aminopropyltriethoxysilane (APTES, 99%, Sigma-Aldrich) by the reflux method. 20 mg of GO was first dispersed in 100 ml of toluene (99.8%, Sigma-Aldrich). The GO dispersed solution was degassed using nitrogen gas (99.995%) for 15 min to remove oxygen within the solution, then 0.6 ml of APTES was injected into the mixed solution. The solution was stirred for 3 hr at 303 K in a nitrogen atmosphere and then refluxed at 383 K for 10 hr under an inert nitrogen gas environment. The APTES grafted-GO (APTES-GO) was rinsed several times with toluene, ethanol and DI water, using an ultracentrifuge.

SiO₂@APTES-GO Composite Preparation:

Each material was dispersed in aqueous solution using 20 mg of APTES-GO, 4 mg of SiO₂ particles and DI water separately. The APTES-GO solution was dropped into the SiO₂ dispersed solution under ultra-sonication, then stored for 24 hr. The coagulated SiO₂@APTES-GO composite was rinsed using an ultracentrifuge with DI water several times.

Probe Immobilization

An oligonucleotide primer was designed and immobilized on the material as a probe for DNA hybridization and its sequence is 5′-GGT-TGG-ATG-CGC-GCA-TCT-ATT-CTG-ACC-CAC-TGG-3′ (SEQ ID NO:1).

### Example 2: A Functionalized Graphene Based 3-Dimensional Structured Material for RNA Detection

### Material Preparation of Example 2

SiO₂ Particle Preparation:

Silica particles were prepared by the modified Stober method in the same manner as in Synthesis Example 1,

Graphene Oxide Synthesis:

The graphene oxide (GO) sheets were prepared in the same manner as in Synthesis Example 1.

Positively Charged Graphene Oxide Preparation:

Positively charged graphene oxide was prepared in Synthesis Example 1.

SiO₂@APTES-GO Composite Preparation:

The SiO₂@APTES-GO composite was prepared in the same manner as in Synthesis Example 1.

Probe Immobilization

An oligonucleotide primer was designed and immobilized on the material as a probe for RNA hybridization and its sequence is 5′-ATA-CAA-TGT-GGC-ATG-TCA-CAC-GTG-GCG-3′ (SEQ ID NO:2).

Dengue Serotype 2 RNA Preparation

The complementary RNA was extracted from dengue virus infected mosquito cell lines. C6/36 cells infected with dengue virus strain 16681 at an MOI of 2 after 70-80% of confluence cell growing. The infected cells were washed using 1×PBS solution and Trizol extraction was performed using 1 ml of Trizol LS reagent. A volume of 0.25 ml Chloroform was added to the homogenized sample, then incubated at temperatures ranging from 288 to 303K for 5 minutes. The solution was incubated again for 10 minutes at the same temperature after vigorous shaking for 15 seconds. After incubation, the sample was centrifuged at 12,000×g for 15 min. at a temperature of 277K. RNA was precipitated using isopropanol after collecting the upper aqueous phase and incubated for 10 min on ice, thereafter, it was centrifuged at 12,000×g for 10 min. The precipitates were collected and washed using 75% ethanol and 7500×g centrifugation at 277K for 5 minutes. The washed precipitates finally resolved in RNase free water after drying.

### Example 3: Positively Charged Particle for DNA Detection

SiO₂ Particle Preparation:

Silica particles were prepared by the modified Stober method in the same manner as in Synthesis Example 1,

Positively Charged SiO₂ Particle Preparation:

APTES-grafted SiO₂ particles were prepared through the reflux method, similarly to the preparation of APTES-GO. An amount of 0.1 g of SiO₂ particles and 150 ml of ethanol were mixed together using ultra-sonication for 30 min, and then the solution was degassed using nitrogen gas. A volume of 0.5 ml of APTES was injected into the degassed solution under nitrogen gas atmosphere and the mixture solution was refluxed at 353 K for 6 hrs. Afterwards, the functionalized SiO₂ was washed several times with ethanol and DI water using an ultracentrifuge.

Probe Immobilization

An oligonucleotide primer was designed and immobilized on the material as a probe for DNA hybridization and its sequence is 5′-GGT-TGG-ATG-CGC-GCA-TCT-ATT-CTG-ACC-CAC-TGG-3′ (SEQ ID NO:1).

### Example 4: Positively Charged Particle for RNA Detection

SiO₂ Particle Preparation:

Silica particles were prepared by the modified Stober method in the same manner as in Synthesis Example 1.

Positively Charged SiO₂ Particle Preparation:

Positively charged Silica particles were prepared in the same manner as in Synthesis Example 2.

Probe Immobilization

An oligonucleotide primer was designed and immobilized on the material as a probe for RNA hybridization and its sequence is 5′-ATA-CAA-TGT-GGC-ATG-TCA-CAC-GTG-GCG-3′ (SEQ ID NO:2).

Dengue Serotype 2 RNA Preparation

The complementary RNA was extracted from dengue virus infected mosquito cell lines in the same manner as in Synthesis Example 2.

### Example 5. Label-Free Zika RNA Impedimetric Biosensors

SiO₂ Particle Preparation:

Silica particles were prepared by the modified Stober method in the same manner as in Synthesis Example 1.

Positively Charged SiO₂ Particle Preparation:

Positively charged Silica particles were prepared in the same manner as in Synthesis Example 2.

Probe Immobilization

An oligonucleotide primer was designed and immobilized on the material as a probe for RNA hybridization and its sequence is AAC-CTT-CGC-TCT-ATT-CTC-ATC-AGT-TTC-ATG (SEQ ID NO:3)

### Comparative Example 1: A Functionalized Graphene Based 2-Dimensional Sheet for DNA Detection

Graphene Oxide Synthesis:

The graphene oxide (GO) sheets were prepared in the same manner as in Synthesis Example 1.

Positively Charged Graphene Oxide Preparation:

Positively charged graphene oxide was prepared in Synthesis Example 1.

Probe Immobilization

An oligonucleotide primer was designed and immobilized on the material as a probe for DNA hybridization and its sequence is 5′-GGT-TGG-ATG-CGC-GCA-TCT-ATT-CTG-ACC-CAC-TGG-3′ (SEQ ID NO:1).

### Evaluation Example 1

### Zeta Potential Analysis

The surface charge was measured using a Malvern Zetasizer (Nano Z, Malvern, UK). We employed 0.02 wt % of material dispersed DI water solution to check Zeta (ζ) potential. The Smoluchowski model was used in order to convert from the electrophoretic mobility to ζ potential.

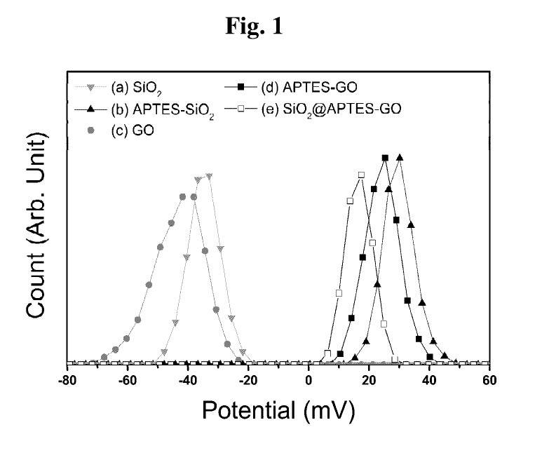

All the functionalized materials showed positive charge due to the presence of amine groups on the surface of the materials (FIG. 1). The average values were +24.4 mV for the material of COMPARATIVE EXAMPLE 1, −34.7 mV for the materials of EXAMPLE 3 and EXAMPLE 4, and +16.7 mV for the materials of EXAMPLE 1 and EXAMPLE 2.

### Evaluation Example 2

### TEM Analysis

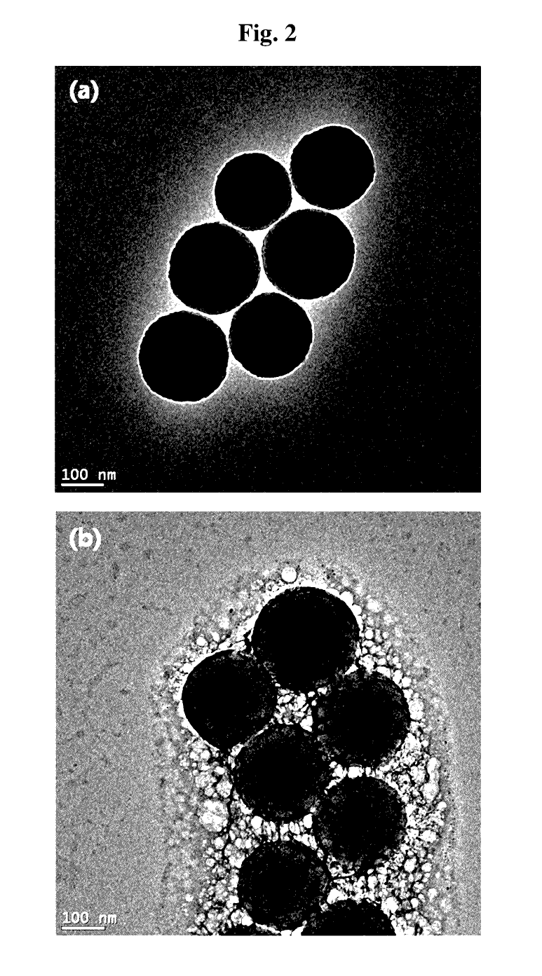

The structure of SiO2 particle and SiO₂@APTES-GO composite particle were observed using a FEI-Tecnai Transmission Electron Microscope (TEM).
FIGS. 2 (a) and (b) clearly shows the microstructure of SiO₂ particles and the Functionalized Graphene Based 3-Dimensional Structured Material. The average size of SiO₂ was 206 nm (FIG. 2a) and the FIG. 2b shows the wrapped structure by the positively charged graphene oxide sheets on the SiO2 particle.

### Evaluation Example 3

Impedance analysis for target DNA or RNA detection.

Biosensor Platform Fabrication:

A 5 mm platinum electrode was first cleaned by polishing with alumina paste and washing by sonication with DI and ethanol solution, and subsequently used for biosensor platform fabrication. A concentration of 0.2 wt. % of the positively functionalized material (APTES-SiO₂, APTES-GO, and SiO₂@APTES-GO) solution was prepared using DI water. A volume of 20 ul of the mixture was dropped on the Pt electrode, and then residual materials were removed by washing with DI water after 10 min. The primer was immobilized on the positively functionalized material layer at room temperature; excessive primers that were not successfully immobilized were removed after primer immobilization, 40 minutes for DNA target and 2 hrs for RNA target separately, through rinsing with DI water.

### Incubation Conditions

The electrodes were incubated with various concentrations (10 pM, 1 fM, and 1 aM) of complementary DNA and 10 pM non-complementary DNA in a 10 mM PBS solution at 333 K for 5 hrs. Finally, the electrodes were washed with DI water to remove unhybridized DNA. RNA hybridization was separately conducted under same condition with that of DNA hybridization under the various concentration of RNA in RNase free water.

Electrochemical Characterization:

Electrochemical impedance spectroscopy (EIS) was performed in 10 mM PBS containing 10 mM K4[Fe(CN)₆]⁴⁻/K3[Fe(CN)₆]³⁻ electrolyte using a Bio-Logic potentiostat (SP-150, Bio-Logic SAS, France). A three-electrode electrochemical cell for EIS analysis was prepared with the as-fabricated biosensor electrode as a working electrode, a Pt wire counter electrode, and an Ag/AgCl reference electrode. Impedance spectra were recorded in the frequency range of 100 mHz to 100 kHz, with 10 mV amplitude. Using the impedance data, the charge-transfer resistance (R_{CT-layer}) of APTES-SiO₂, APTES-GO, and SiO₂@APTES-GO, respectively, of a immobilized primer layer (R_{CT-primer}), and of a hybridized complementary DNA or RNA layer (R_{CT-COM}) were analyzed using the Randles' model (Rushworth and Hirst, 2013) and calculated from subtracting R_{CT} the Probe immobilized electrode, from the RCT after incubation using test solution.

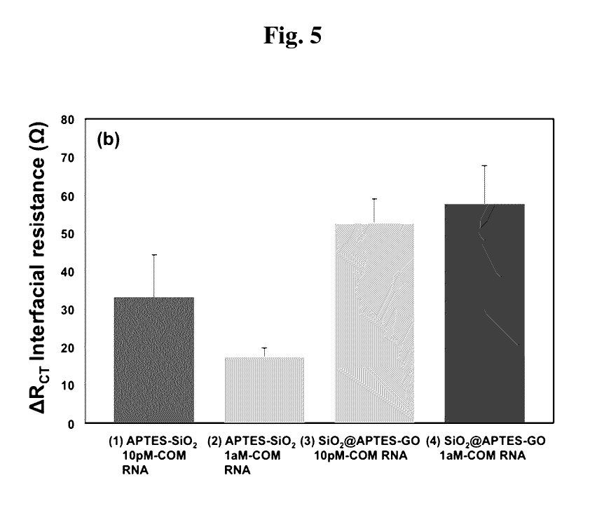

Referring to FIGS. 5, 6 and 7, it is confirmed that all of the particles with positive charge were able to detect complementary target with these values.

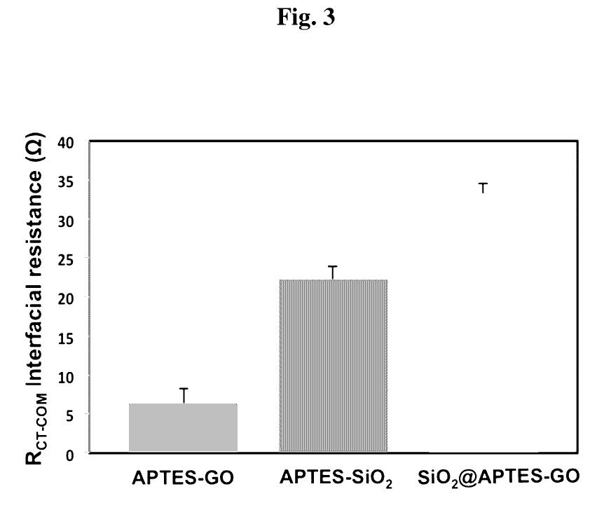

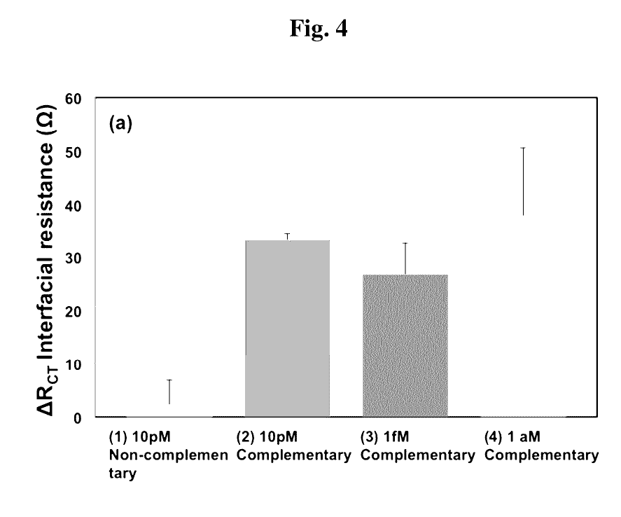

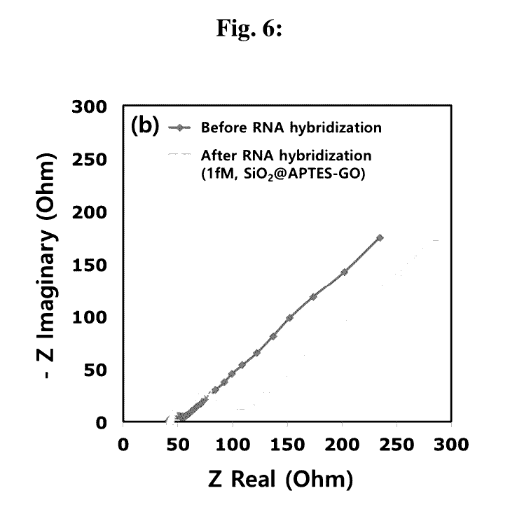

FIG. 3 is showing the charge transfer resistance change from the hybridization of DNA on the probe immobilized surface, R_{CT-COM}. The R_{CT-COM} values for APTES-GO (Comparative EXAMPLE 1), APTES-SiO₂ (EXAMPLE 3), and SiO2@APTES-GO (EXAMPLE 1) are 6.37±1.97 Ω, 22.22±1.7Ω, and 33.29±1.24Ω, respectively.
FIG. 4 represents that the material of EXAMPLE 1 (SiO2@APTES-GO) is able to detect down to latto-molar concentration of Dengue serotype 2 DNA.
FIG. 5 shows the results in terms of after 10 pM and 1 aM RNA hybridization of APTES-SiO₂ and SiO₂@APTES-GO. The R_{CT-COM} values were 30.19±4.02Ω for APTES-SiO₂ (EXAMPLE 4), and 53.72±4.82Ω for SiO₂@APTES-GO (EXAMPLE 2), respectively. All the R_{CT-COM} values were from the half circle diameter increasing after target hybridization (FIG. 6). FIG. 6 represent 1 fM dengue DNA detection result.

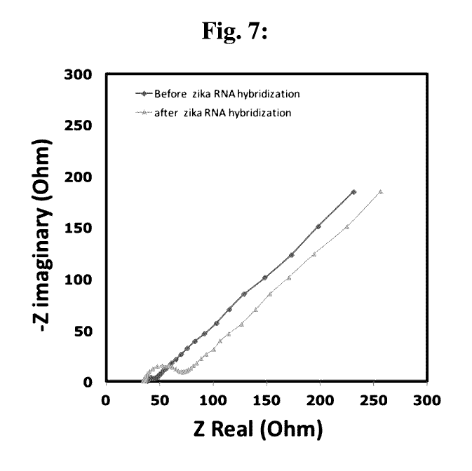

Very recent work showed successful detection of 1 pM Zika RNA by using the same principles of impedance sensing and the same graphene-oxide based electrode materials (FIG. 7). Oligonucleotide probes complementary to ZIKV RNA with the sequence AACCTTCGCTCTATTCTCATCAGTTTCATG (SEQ ID NO:3) have been prepared and used to detect Zika virus of 1 pM concentration. This initial result serves as proof of concept for the biosensing platform feasibility and transferability to Zika detection. The probe immobilized functional particle detected 1 pM Zika RNA (FIG. 7) upon incubation of several hours of the probe with Zika RNA.

We have employed electrochemical impedance spectroscopy (EIS) to demonstrate the working principles of our developed virus bio-sensing technology utilizing commercial laboratory equipment. EIS is a versatile tool that measures the electrical impedance of a system as a function of frequency. It is a versatile technique widely employed in diverse fields such as electrochemistry, medicine, biology, food science, geology, etc. However, conventional EIS methods, in particular those performed in laboratory instruments are very slow at low frequencies, a frequency range often characteristic of the response of biosensors. Thus, an extensive review of the available methods and electronic devices that can perform the key processes pertaining the biosensor platform is required. These parameters include analyte recognition, signal transduction, readout and data transmission. Components and measuring techniques will be selected with a focus on specificity, speed, portability, and low costs. In addition, discrete component will be selected to optimize the detection sensitivity of the Zika biosensor and assembled in a circuit breadboard together with the readout and data transmission electronics; intrinsic noise and environmental interference cancellation will be considered. Point of care bio-sensing requires that the device operates in widely different environmental conditions. Therefore, circuitry providing intrinsic noise cancellation and temperature and humidity fluctuation immunity to signal detection and transduction are imperative. Designs employing resonance circuitry in a Wheatstone bridge configuration will be investigated as a vehicle to provide cancellation of noise sources and signal shifts caused by environmental perturbations such as thermal and humidity fluctuations.

Those skilled in the art will recognize that numerous modifications can be made to the specific implementations described above. The implementations should not be limited to the particular limitations described. Other implementations may be possible.

### REFERENCES

Abdul Rahman, S., Saadun, R., Azmi, N. E., Ariffin, N., Abdullah, J., Yusof, N. A., Sidek, H., Hajian, R., 2014. Label-Free Dengue Detection Utilizing PNA/DNA Hybridization Based on the Aggregation Process of Unmodified Gold Nanoparticles. J. Nanomater. 2014, e839286. doi:10.1155/2014/839286
Ahmadi, H., Heidarzadeh, H., Taghipour, A., Rostami, A., Baghban, H., Dolatyari, M., Rostami, G., 2014. Evaluation of single virus detection through optical biosensor based on microsphere resonator. Opt.-Int. J. Light Electron Opt. 125, 3599-3602. doi:10.1016/j.ijleo.2014.01.087
Bahsi, Z. B., Büyükaksoy, A., Ölmezcan, S. M., [symbol] im [symbol] ek, F., Aslan, M. H., Oral, A. Y., 2009. A Novel Label-Free Optical Biosensor Using Synthetic Oligonucleotides from E. coli O157:H7: Elementary Sensitivity Tests. Sensors 9, 4890-4900. doi:10.3390/s90604890
Bracewell, D. G., Brown, R. A., Hoare, M., 2004. Addressing a whole bioprocess in real-time using an optical biosensor-formation, recovery and purification of antibody fragments from a recombinant E. coli host. Bioprocess Biosyst. Eng. 26, 271-282. doi:10.1007/s00449-004-0359-z
Cheng, M. S., Toh, C.-S., 2013. Novel biosensing methodologies for ultrasensitive detection of viruses. Analyst 138, 6219-6229. doi:10.1039/C3AN01394D
Deng, J., Toh, C.-S., 2013. Impedimetric DNA Biosensor Based on a Nanoporous Alumina Membrane for the Detection of the Specific Oligonucleotide Sequence of Dengue Virus. Sensors 13, 7774-7785. doi:10.3390/s130607774
Feng, X., Chen, N., Zhang, Y., Yan, Z., Liu, X., Ma, Y., Shen, Q., Wang, L., Huang, W., 2014. The self-assembly of shape controlled functionalized graphene-MnO2 composites for application as supercapacitors. J. Mater. Chem. A 2, 9178. doi:10.1039/c3ta15402e
Forrow, N. J., Bayliff, S. W., 2005. A commercial whole blood glucose biosensor with a low sensitivity to hematocrit based on an impregnated porous carbon electrode. Biosens. Bioelectron. 21, 581-587. doi:10.1016/j.bios.2004.12.011
Forrow, N. J., Sanghera, G. S., Walters, S. J., Watkin, J. L., 2005. Development of a commercial amperometric biosensor electrode for the ketone d-3-hydroxybutyrate. Biosens. Bioelectron., Selected Papers from the Eighth World Congress on Biosensors, Part I 20, 1617-1625. doi:10.1016/j.bios.2004.07.009
Guzman, M. G., Harris, E., 2015. Dengue. The Lancet 385, 453-465. doi:10.1016/50140-6736(14)60572-9
Guzman, M. G., Vázquez, S., Kouri, G., 2009. Dengue: where are we today? Malays. J. Med. Sci. MJMS 16, 4-11.
Hu, C., Lu, T., Chen, F., Zhang, R., 2013. A brief review of graphene-metal oxide composites synthesis and applications in photocatalysis. J. Chin. Adv. Mater. Soc. 1, 21-39. doi:10.1080/22243682.2013.771917
Hummers, W. S., Offeman, R. E., 1958. Preparation of Graphitic Oxide. J. Am. Chem. Soc. 80, 1339-1339. doi:10.1021/ja01539a017
Idrees, S., Ashfaq, U. A., 2012. A brief review on dengue molecular virology, diagnosis, treatment and prevalence in Pakistan. Genet. Vaccines Ther. 10, 1-10. doi:10.1186/1479-0556-10-6
Kuila, T., Bose, S., Khanra, P., Mishra, A. K., Kim, N. H., Lee, J. H., 2011. Recent advances in graphene-based biosensors. Biosens. Bioelectron. 26, 4637-4648. doi:10.1016/j.bios.2011.05.039
Li, Z.-F., Zhang, H., Liu, Q., Liu, Y., Stanciu, L., Xie, J., 2014. Novel Pyrolyzed Polyaniline-Grafted Silicon Nanoparticles Encapsulated in Graphene Sheets As Li-Ion Battery Anodes. ACS Appl. Mater. Interfaces 6, 5996-6002. doi:10.1021/am501239r
Long, F., Zhu, A., Shi, H., 2013. Recent Advances in Optical Biosensors for Environmental Monitoring and Early Warning. Sensors 13, 13928-13948. doi:10.3390/s131013928
Lu, C.-H., Yang, H.-H., Zhu, C.-L., Chen, X., Chen, G.-N., 2009. A Graphene Platform for Sensing Biomolecules. Angew. Chem. Int. Ed. 48, 4785-4787. doi:10.1002/anie.200901479
Myung, S., Solanki, A., Kim, C., Park, J., Kim, K. S., Lee, K.-B., 2011. Graphene-Encapsulated Nanoparticle-Based Biosensor for the Selective Detection of Cancer Biomarkers. Adv. Mater. 23, 2221-2225. doi:10.1002/adma.201100014
Nascimento, H. P. O., Oliveira, M. D. L., de Melo, C. P., Silva, G. J. L., Cordeiro, M. T., Andrade, C. A. S., 2011. An impedimetric biosensor for detection of dengue serotype at picomolar concentration based on gold nanoparticles-polyaniline hybrid composites. Colloids Surf. B Biointerfaces 86, 414-419. doi:10.1016/j.colsurfb.2011.04.028
Ou, J., Wang, J., Liu, S., Mu, B., Ren, J., Wang, H., Yang, S., 2010. Tribology Study of Reduced Graphene Oxide Sheets on Silicon Substrate Synthesized via Covalent Assembly. Langmuir 26, 15830-15836. doi:10.1021/1a102862d
Rai, V., Hapuarachchi, H. C., Ng, L. C., Soh, S. H., Leo, Y. S., Toh, C.-S., 2012. Ultrasensitive cDNA Detection of Dengue Virus RNA Using Electrochemical Nanoporous Membrane-Based Biosensor. PLoS ONE 7, e42346. doi:10.1371/journal.pone.0042346
Rinken, T. (Ed.), 2013. State of the Art in Biosensors—General Aspects. InTech.
Rushworth, J. V., Hirst, N. A., 2013. Impedimetric Biosensors for Medical Applications: Current Progress and Challenges. Momentum Press.
Sabchareon, A., Wallace, D., Sirivichayakul, C., Limkittikul, K., Chanthavanich, P., Suvannadabba, S., Jiwariyavej, V., Dulyachai, W., Pengsaa, K., Wartel, T. A., Moureau, A., Saville, M., Bouckenooghe, A., Viviani, S., Tornieporth, N. G., Lang, J., 2012. Protective efficacy of the recombinant, live-attenuated, CYD tetravalent dengue vaccine in Thai schoolchildren: a randomised, controlled phase 2b trial. The Lancet 380, 1559-1567. doi:10.1016/S0140-6736(12)61428-7
Singal, S., Biradar, A. M., Mulchandani, A., Rajesh, 2014. Ultrasensitive Electrochemical Immunosensor Based on Pt Nanoparticle-Graphene Composite. Appl. Biochem. Biotechnol. 1-13. doi:10.1007/s12010-014-0933-z
Stankovich, S., Dikin, D. A., Dommett, G. H. B., Kohlhaas, K. M., Zimney, E. J., Stach, E. A., Piner, R. D., Nguyen, S. T., Ruoff, R. S., 2006. Graphene-based composite materials. Nature 442, 282-286. doi:10.1038/nature04969
Stringer, R. C., Schommer, S., Hoehn, D., Grant, S. A., 2008. Development of an optical biosensor using gold nanoparticles and quantum dots for the detection of Porcine Reproductive and Respiratory Syndrome Virus. Sens. Actuators B Chem. 134, 427-431. doi:10.1016/j.snb.2008.05.018
Sun, H., Sun, X., Hu, T., Yu, M., Lu, F., Lian, J., 2014. Graphene-Wrapped Mesoporous Cobalt Oxide Hollow Spheres Anode for High-Rate and Long-Life Lithium Ion Batteries. J. Phys. Chem. C 118, 2263-2272. doi:10.1021/jp408021m
Teles, F. R. R., Fonseca, L. P., 2008. Trends in DNA biosensors. Talanta 77, 606-623. doi:10.1016/j.talanta.2008.07.024
Teles, F. S. R. R., 2011. Biosensors and rapid diagnostic tests on the frontier between analytical and clinical chemistry for biomolecular diagnosis of dengue disease: A review. Anal. Chim. Acta 687, 28-42. doi:10.1016/j.aca.2010.12.011
Wang, L., Hua, E., Liang, M., Ma, C., Liu, Z., Sheng, S., Liu, M., Xie, G., Feng, W., 2014. Graphene sheets, polyaniline and AuNPs based DNA sensor for electrochemical determination of BCR/ABL fusion gene with functional hairpin probe. Biosens. Bioelectron. 51, 201-207. doi:10.1016/j.bios.2013.07.049
Wei, D., Liang, J., Zhu, Y., Yuan, Z., Li, N., Qian, Y., 2013. Formation of Graphene-Wrapped Nanocrystals at Room Temperature through the Colloidal Coagulation Effect. Part. Part. Syst. Charact. 30, 143-147. doi:10.1002/ppsc.201200120
Wilder-Smith, A., Gubler, D. J., 2008. Geographic Expansion of Dengue: The Impact of International Travel. Med. Clin. North Am., New and Emerging Infectious Diseases 92, 1377-1390. doi:10.1016/j.mcna.2008.07.002
Wu, Z.-S., Zhou, G., Yin, L.-C., Ren, W., Li, F., Cheng, H.-M., 2012. Graphene/metal oxide composite electrode materials for energy storage. Nano Energy 1, 107-131. doi:10.1016/j.nanoen.2011.11.001
Xu, S., 2012. Electromechanical biosensors for pathogen detection. Microchim. Acta 178, 245-260. doi:10.1007/s00604-012-0831-4
Yan, R., Lynn, N. S., Kingry, L. C., Yi, Z., Erickson, T., Slayden, R. A., Dandy, D. S., Lear, K. L., 2012. Detection of virus-like nanoparticles via scattering using a chip-scale optical biosensor. Appl. Phys. Lett. 101, 161111. doi:10.1063/1.4758294
Zhang, W., Asiri, A. M., Liu, D., Du, D., Lin, Y., 2014. Nanomaterial-based biosensors for environmental and biological monitoring of organophosphorus pesticides and nerve agents. TrAC Trends Anal. Chem. 54, 1-10. doi:10.1016/j.trac.2013.10.007

1. A biosensor platform configured to provide impedimetric data in the presence of a virus DNA or RNA, comprising:
an electrode material coupled to at least one functionalized particle;
a supporting membrane, wherein the electrode material is disposed on the supporting membrane; and
a label-free virus DNA or RNA immobilized to said electrode material, wherein said DNA or RNA is complementary to said virus DNA or RNA.

2. The biosensor platform of claim 1, wherein the supporting membrane comprises a rigid substrate.

3. The biosensor platform of claim 1, wherein the functionalized material comprises graphene.

4. The biosensor platform of claim 1, wherein the functional group has a positive charge.

5. The biosensor platform of claim 1, wherein said virus DNA or RNA is selected from dengue, yellow fever, chikungunya, West Nile and Zika virus.

6. A biosensor array comprises a plurality of functionalized three-dimensional material, wherein said material is incorporated with at least one nucleotide primer for label-free virus DNA and RNA detection based on impedimetric data collection.

7. A biosensor platform to detect at least one vector-borne virus, comprising:
a functionalized electrode surface;
at least one nucleotide primer immobilized on the functionalized electrode surface, wherein said nucleotide primer is diagnostic for said at least one vector borne virus DNA or RNA; and
an electrochemical impedance spectroscope (EIS), wherein said EIS is configured to measure the impedance change upon said primer hybridization to said virus DNA or RNA.

8. The biosensor platform according to claim 7 wherein said functionalized electrode surface comprises an electrode material deposited on a supporting membrane.

9. The biosensor platform according to claim 7 wherein said electrode material is silicon dioxide.

10. The biosensor platform according to claim 7, wherein said supporting membrane is a functionalized graphene sheet.

11. The biosensor platform according to claim 7, wherein said functionalized electrode surface is 3-Aminopropyltriethoxysilane (APTES) functionalized graphene oxide (APTES-GO) wrapped SiO₂ particle composite (SiO₂@APTES-GO).

12. The biosensor platform according to claim 7, further comprises a microfluidics device to extract said at least one vector borne virus's nucleotides for hybridization.

13. A method for detecting and monitoring insect-borne viruses, comprising:
a. placing a biosensor device in a predetermined location, wherein said biosensor device is pre-loaded with at least one specific primer or probe for at least one insect-borne virus;
b. obtaining at least one sample;
c. placing the sample on the biosensor device, wherein the biosensor device is configured to measure changes in impedance to an applied electrical current;
d. identifying at least one sample with impedance increase after the at least one sample is placed on the biosensor device, wherein said impedance increase indicates the presence of said at least one insect-borne virus; and
e. transmitting the result of step d via a transmitting device to a central facility.

14. The method of claim 13, wherein the at least one insect is an infected arthropod species selected from the group consisting of mosquitoes, ticks, triatomine bugs, sandflies and backflies.

15. A method for detecting at least one vector borne virus, comprising:
a. Providing a biosensor platform comprising at least one moiety that is immobilized on a functionalized electrode surface, said moiety is diagnostic for at least one vector borne virus;
b. Contacting said biosensor platform with a sample;
c. Observing said vector borne virus specific impedance change to identify the presence of said at least one vector borne virus.

16. The method of claim 15 is configured for point of care detection with additional wireless data transmission device, power source and global position system to transmit the virus infection data in said predetermined location.

17. The method of claim 15, wherein the sample is from a human blood, urine or saliva, wherein the human having been bitten by an infected arthropod.

18. The method of claim 15 is to monitor insect-borne viruses infected populations.

19. A method for detection of nucleic acid (NA), comprising:
a. Providing a biosensor platform comprising at least one nucleotide primer immobilized on a functionalized electrode surface, said nucleotide primer is diagnostic for at least one NA;
b. Contacting said biosensor platform with a sample;
c. Observing said NA specific impedance change to identify the presence of NA.

20. The method of claim 19, wherein the NA is DNA or RNA.
